(19) United States
(12) Patent Application Publication
(10) Pub. No.: US 2005/0182453 A1
(43) Pub. Date: Aug. 18, 2005

(54) Treatment of epilepsy by high frequency electrical stimulation and/or drug stimulation

(76) Inventors: Todd K. Whitehurst, Santa Clarita, CA (US); Edward H. Overstreet, Valencia, CA (US); Albert A. Maltan, Sistrans (AT)

Correspondence Address: STEVEN L. NICHOLS;RADER, FISHMAN & GRAVER PLLC, 10653 S. RIVER FRONT PARKWAY, SUITE 150, SOUTH JORDAN, UT, 84095, US

(21) Appl. No.: 11/103,945

(22) Filed: Apr. 12, 2005

Related U.S. Application Data
(63) Continuation-in-part of application No. 10/428,743, filed on May 2, 2003.
(60) Provisional application No. 60/383,317, filed on May 24, 2002.

(51) Int. Cl. A61N 1/18

(52) U.S. Cl. 607/45

(57) ABSTRACT

Exemplary methods of treating a patient with epilepsy include applying a stimulus to a stimulation site within the patient with an implanted system control unit in accordance with one or more stimulation parameters. The stimulus includes a stimulation current having a frequency substantially equal to or greater than 400 Hz. Exemplary systems for treating a patient with epilepsy include a system control unit configured to apply a stimulus to a stimulation site within the patient in accordance with one or more stimulation parameters. The stimulus includes a stimulation current having a frequency substantially equal to or greater than 400 Hz.

### RELATED APPLICATIONS

The present application is a continuation-in-part application of U.S. application Ser. No. 10/428,743, filed May 2, 2003, which application claims the benefit of Provisional Application Ser. No. 60/383,317, filed May 24, 2002. Both applications are incorporated herein by reference in their entireties.

### BACKGROUND

Epilepsy is characterized by a tendency to recurrent seizures that can lead to loss of awareness, loss of consciousness, and/or disturbances of movement, autonomic function, sensation (including vision, hearing and taste), mood, and/or mental function. Epilepsy afflicts 1-2 percent of the population in the developed world. The mean prevalence of active epilepsy (i.e., continuing seizures or the need for treatment) in developed and undeveloped countries combined is estimated to be 7 per 1,000 of the general population, or approximately 40 million people worldwide. Studies in developed countries suggest an annual incidence of epilepsy of approximately 50 per 100,000 of the general population. However, studies in developing countries suggest this figure is nearly double at 100 per 100,000.

The primary pathology of epilepsy is a synchronization of electrical activity between large numbers of brain neurons. Neurons “fire”, i.e., transmit an electrical depolarization pulse down an axon(s), multiple times per second. While a group of adjacent neurons may normally demonstrate some correlation in their firing pattern, they normally do not all fire with exactly the same rate and exactly the same timing. However, during a seizure, a group of neurons in the brain demonstrate a highly synchronized firing pattern. This group may be localized, in which case it may be referred to as the seizure focus. In some types of epilepsy, the focus may remain fixed. In other types of epilepsy, the patient may have multiple fixed foci, and a seizure may arise from any of the foci. In still other types of epilepsy, a seizure may arise from a seemingly random location. Finally, in some types of epilepsy a seizure appears to arise from a majority of the brain all at once, i.e., with no focus. Seizures that arise from a focus may remain localized, in which case the symptoms of the seizure depend on the site of the focus. Seizures that arise from a focus may also spread to the majority of the brain, i.e., they may be initially focal but become secondarily generalized.

Epilepsy is often, but not always, the result of underlying brain disease. Any type of brain disease can cause epilepsy, but not all patients with the same brain pathology will develop epilepsy. The cause of epilepsy cannot be determined in a number of patients; however, the most commonly accepted theory posits that epilepsy is the result of an imbalance of certain chemicals in the brain, e.g., neurotransmitters. Children and adolescents are more likely to have epilepsy of unknown or genetic origin. The older the patient, the more likely it is that the cause is an underlying brain disease such as a brain tumor or cerebrovascular disease.

Trauma and brain infection may cause epilepsy at any age, and in particular, account for the higher incidence rate of epilepsy in developing countries. For example, in Latin America, neurocysticercosis (cysts on the brain caused by tapeworm infection) is a common cause of epilepsy. In Africa, AIDS and its related infections, malaria, and meningitis are common causes of epilepsy. In India, AIDS, neurocysticercosis, and tuberculosis are common causes of epilepsy. Febrile illness of any kind, whether or not it involves the brain, may trigger seizures in vulnerable young children and cause the children to develop epilepsy later in life.

### SUMMARY

Exemplary methods of treating a patient with epilepsy include applying a stimulus to a stimulation site within the patient with an implanted system control unit in accordance with one or more stimulation parameters. The stimulus includes a stimulation current having a frequency substantially equal to or greater than 400 Hz.

Exemplary systems for treating a patient with epilepsy include a system control unit configured to apply a stimulus to a stimulation site within the patient in accordance with one or more stimulation parameters. The stimulus includes a stimulation current having a frequency substantially equal to or greater than 400 Hz.

### BRIEF DESCRIPTION OF THE DRAWINGS

The accompanying drawings illustrate various embodiments of the present invention and are a part of the specification. The illustrated embodiments are merely examples of the present invention and do not limit the scope of the invention.

FIG. 1A depicts the dorsal surface of the brain stem according to principles described herein.

FIG. 1B is a section view through the brain stem depicted in FIG. 1A according to principles described herein.

FIG. 1C is another section view through the brain stem depicted in FIG. 1A according to principles described herein.

FIG. 2A depicts the lateral surface of the brain according to principles described herein.

FIG. 2B depicts the medial surface of the head according to principles described herein.

FIGS. 2C-2F depict coronal section views of the brain of FIG. 2B according to principles described herein.

FIGS. 3A, 3B, and 3C show some possible configurations of an implantable microstimulator system control unit (SCU) according to principles described herein.

FIG. 4 shows an SCU that has been implanted beneath the scalp of a patient according to principles described herein.

FIG. 5 shows that the implanted SCU may be configured to communicate with a number of external devices according to principles described herein.

FIG. 6 depicts a number of implantable devices configured to communicate with each other and/or with one or more external devices according to principles described herein.

Throughout the drawings, identical reference numbers designate similar, but not necessarily identical, elements.

### DETAILED DESCRIPTION

Methods and systems for treating a patient with epilepsy are described herein. A system control unit (SCU) is implanted within the patent. The SCU is configured to apply a stimulus to a stimulation site within the patient in accordance with one or more stimulation parameters. The stimulus includes a stimulation current having a frequency substantially equal to or greater than 400 Hertz (Hz). The stimulus may additionally or alternatively include an infusion of one or more drugs into the stimulation site.

In the following description, for purposes of explanation, numerous specific details are set forth in order to provide a thorough understanding of the present systems and methods. It will be apparent, however, to one skilled in the art that the present systems and methods may be practiced without these specific details. Reference in the specification to “one embodiment” or “an embodiment” means that a particular feature, structure, or characteristic described in connection with the embodiment is included in at least one embodiment. The appearance of the phrase “in one embodiment” in various places in the specification are not necessarily all referring to the same embodiment.

The following description is of the best mode presently contemplated for carrying out the invention. This description is not to be taken in a limiting sense, but is made merely for the purpose of describing the general principles of the invention. The scope of the invention should be determined with reference to the claims.

Recent studies in both developed and developing countries have shown that up to 70 percent of newly diagnosed children and adults with epilepsy can be successfully treated (i.e., complete control of seizures for several years) with anti-epileptic drugs. After two to five years of successful treatment, drugs can be withdrawn in about 70 percent of children and 60 percent of adults without the patient experiencing relapses. However, up to 30 percent of patients are refractory to medication. There is evidence that the longer the history of epilepsy, the harder it is to control. The presence of an underlying brain disease typically results in a worse prognosis in terms of seizure control. Additionally, partial seizures, especially if associated with brain disease, are more difficult to control than generalized seizures.

Pharmacological agents for the treatment of epilepsy typically work by suppressing neural activity. For example, some epilepsy drugs appear to increase the threshold voltage at which a neuron may fire. These medications thus typically have sedative side effects. Other medications have significant negative side effects, including potential cognitive deficits.

Patients suffering from epilepsy may undergo surgery to remove a part of the brain in which the seizures are believed to arise, i.e., the seizure focus. However, in many patients a seizure focus cannot be identified, and in others the focus is in an area that cannot be removed without significant detrimental impact on the patient. For example, in temporal lobe epilepsy, patients may have a seizure focus in the hippocampi bilaterally. However, both hippocampi cannot be removed without adversely affecting a patient's long-term memory. Other patients may have a seizure focus that lies adjacent to a critical area such as the speech center.

Vagus nerve stimulation (VNS) has been applied with partial success in patients with refractory epilepsy. In this procedure, an implantable pulse generator (IPG) is implanted in the patient's thorax, and an electrode lead is routed from the IPG to the left vagus nerve in the neck. Helix-shaped stimulation and indifferent electrodes are attached to the vagus nerve via an invasive surgical process that requires the carotid sheath to be fully exposed. Based on a number of studies, approximately five percent of patients undergoing VNS are seizure-free, and an additional 30-40 percent of patients have a greater than 50 percent reduction in seizure frequency.

In addition to this relatively low efficacy, VNS may lead to significant side effects. The vagus nerve provides parasympathetic innervation to the cardiac tissue, and thus VNS may lead to bradycardia, arrhythmia, or even graver cardiac side effects. In fact, VNS systems may only be used on the left vagus nerve, as the right vagus nerve contributes significantly more to cardiac innervation. Additionally, VNS may interfere with proper opening of the vocal cords, which has led to hoarseness and shortness of breath in a significant number of VNS patients.

The exact mechanism of seizure suppression using VNS is unknown. The nucleus of tractus solitarius (NTS; a.k.a., nucleus of the solitary tract) is a primary site at which vagal afferents terminate. Because afferent vagal nerve stimulation has been demonstrated to have anticonvulsant effects, it is likely that changes in synaptic transmission in the NTS can regulate seizure susceptibility. To demonstrate this, Walker, et al. (“Regulation of limbic motor seizures by GABA and glutamate transmission in nucleus tractus solitarius,” Epilepsia, 1999 August) applied muscimol, an agonist of the inhibitory neurotransmitter GABA, to the NTS in a murine model of epilepsy. Muscimol applied to the NTS attenuated seizures in all seizure models tested, whereas muscimol applied to adjacent regions of NTS had no effect. Additionally, bicuculline methiodide, a GABA antagonist, injected into the NTS did not alter seizure responses. Finally, anticonvulsant effects were also obtained with application of lidocaine, a local anesthetic, into the NTS. Unilateral injections were sufficient to afford seizure protection. Walker, et al. concludes that inhibition of the NTS outputs enhances seizure resistance in the forebrain and provides a potential mechanism for the seizure protection obtained with vagal stimulation.

The NTS sends fibers bilaterally to the reticular formation and hypothalamus, which are important in the reflex control of cardiovascular, respiratory, and gastrointestinal functions. The NTS also provides input to the dorsal motor nucleus of the vagus, which enables the parasympathetic fibers of the vagus nerve to control these reflex responses. The NTS runs the entire length of the medulla oblongata, and the NTS (as well as the trigeminal nuclei) receives somatic sensory input from all cranial nerves, with much of its input coming from the vagus nerve.

A significant number of neurons in the trigeminal nerve may project to the NTS. After applying horseradish peroxidase to peripheral branches of the trigeminal nerve in a cat, Nomura, et al. found that branches of the trigeminal nerve (the lingual and pterygopalatine nerves) were found to contain fibers which ended ipsilaterally in the rostral portions of the NTS: massively in the medial and ventrolateral NTS, moderately in the intermediate and interstitial NTS, and sparsely in the ventral NTS. (The rostralmost part of the NTS was free from labeled terminals.) After injecting the enzyme into the NTS portions rostral to the area postrema, small neurons were scattered in the maxillary and mandibular divisions of the trigeminal ganglion. The authors concluded that trigeminal primary afferent neurons project directly to the NTS. [See Nomura, et al. “Trigeminal primary afferent neurons projecting directly to the solitary nucleus in the cat: a transganglionic and retrograde horseradish peroxidase study.” Neurosci Lett 1984 Sep. 7; 50(1-3):257-62.] In another study, by staining for substance P immunoreactivity, South, et al found that Substance P-containing trigeminal sensory neurons project to the NTS. [See South, et al. “Substance P-containing trigeminal sensory neurons project to the nucleus of the solitary tract.” Brain Res 1986 May 7; 372(2):283-9.]

The major brainstem nuclei that serve as the source for the trigeminal nerve are: the motor trigeminal nucleus, found in the midpons; the mesencephalic trigeminal nucleus located in the pons contains primary sensory neurons whose axons carry proprioceptive information from the muscles of mastication; the main (or primary) trigeminal sensory nucleus, the largest of the cranial nerve nuclei, which extends from the midbrain down to the second cervical segment of the spinal cord; and the spinal (or descending) trigeminal nucleus, which extends from the main trigeminal sensory nucleus to the dorsal gray of the spinal cord and contains secondary sensory neurons that process pain and temperature information.

A significant number of neurons in the trigeminal nuclei may also project to the NTS. Menetrey, et al used the retrograde transport of a protein-gold complex to examine the distribution of spinal cord and trigeminal nucleus caudalis neurons that project to the NTS in the rat. [See Menetrey, et al. “Spinal and trigeminal projections to the nucleus of the solitary tract: a possible substrate for somatovisceral and viscerovisceral reflex activation.” J Comp Neurol 1987 Jan. 15; 255(3):439-50.] The authors found that retrogradely labeled cells were numerous in the superficial laminae of the trigeminal nucleus caudalis, through its rostrocaudal extent. Since the NTS is an important relay for visceral afferents from both the glossopharyngeal and vagus nerves, the authors suggest that the spinal and trigeminal neurons that project to the NTS may be part of a larger system that integrates somatic and visceral afferent inputs from wide areas of the body. The projections may underlie somatovisceral and/or viscerovisceral reflexes, perhaps with a significant afferent nociceptive component.

Beart, et al utilized microinfusion and retrograde transport of D-[3H]aspartate to identify excitatory afferents to the NTS. [See Beart, et al. “Excitatory amino acid projections to the nucleus of the solitary tract in the rat: a retrograde transport study utilizing D-[3H]aspartate and [3H]GABA.” J Auton Nerv Syst 1994 Dec. 1; 50(1):109-22.] The authors found that the heaviest labeling was localized bilaterally in the trigeminal nucleus with cells extending through its subdivisions and the entire rostrocaudal axis.

The trigeminal nerve contributes a significant number of afferent fibers to the NTS. Additionally, trigeminal nerve afferents synapse on the trigeminal nucleus in the brainstem, and afferents from the trigeminal nucleus also project to the NTS. Thus, electrical stimulation of one or ore of the trigeminal nuclei may reasonably be expected to demonstrate efficacy in the treatment of patients with medically refractory epilepsy. In fact, Fanselow, et al. recently demonstrated that unilateral stimulation (via a chronically implanted nerve cuff electrode) of the infraorbital branch of the trigeminal nerve led to a reduction in electrographic seizure activity of up to 78 percent; the authors report that bilateral trigeminal stimulation was even more effective. [See Fanselow E E; Reid A P; Nicolelis M A. “Reduction of pentylenetetrazole-induced seizure activity in awake rats by seizure-triggered trigeminal nerve stimulation.” J Neurosci 2000 Nov. 1; 20(21):8160-8.]

To determine the contribution of the locus coeruleus to the anti-epileptic effects of vagus nerve stimulation, Krahl, et al. chemically lesioned the locus coeruleus to determine if it is a critical structure involved in the anticonvulsant mechanisms of VNS (Krahl, et al. “Locus coeruleus lesions suppress the seizure-attenuating effects of vagus nerve stimulation.” Epilepsia 1998 July; 39(7):709-14). Rats were chronically depleted of norepinephrine by a bilateral infusion of 6-hydroxydopamine (6-OHDA) into the locus coeruleus. (The locus coeruleus releases much of the norepinephrine neurotransmitter found in the brain.) Two weeks later, they were tested with maximal electroshock (MES) to assess VNS-induced seizure suppression. In another experiment, the locus coeruleus was acutely inactivated with lidocaine, and seizure suppression was tested in a similar fashion. VNS significantly reduced seizure severities of control rats. However, in animals with chronic or acute locus coeruleus lesions, VNS-induced seizure suppression was attenuated. This data indicates that the locus coeruleus is involved in the circuitry necessary for the anticonvulsant effects of VNS. Seizure suppression by VNS may therefore depend on the release of norepinephrine, a neuromodulator that has anticonvulsant effects. These data suggest that noradrenergic agonists might enhance VNS-induced seizure suppression.

The thalamus is believed to play a major role in some types of epilepsy by acting as a center for seizure onset or as a relay station in allowing a focal seizure to propagate. In a Single Positron Emission Computed Tomography (SPECT) study of patients with left-sided VNS systems, a consistent decrease of activity was found in the left thalamus caused by VNS. The authors concluded that left-sided VNS reduces seizure onset or propagation through inhibition of the thalamic relay center.

Thalamic relay neurons are used in generating 3 Hz absence seizures and are believed to be involved in other types of epilepsy. Thalamic nuclei of some patients suffering from epilepsy display neuronal activities described as “low-threshold calcium spike bursts”, which have been shown to be related to a state of membrane hyperpolarization of thalamic relay neurons. This thalamic rhythmicity is transmitted to the related cortex, thanks to thalamocortical resonant properties. In the cortex, an asymmetrical corticocortical inhibition (edge effect) at the junction between low and high frequency zones is proposed to be at the origin of a cortical activation of high frequency areas bordering low frequency ones.

The “thalamic relay” theory has led researchers recently to begin implanting deep brain stimulation (DBS) systems for stimulation of either the centromedian nucleus or the anterior nucleus of the thalamus, in order to treat medically refractory epilepsy patients. Unfortunately, the efficacy of this invasive procedure has thus far proven to be approximately the same as VNS.

In 1989, Shandra, et al. demonstrated with acute experiments on cats that seizure-related discharges were provoked by relatively low frequency (7-12 Hz) electrical stimulation of the ventrolateral nucleus of the thalamus (Shandra, et al. “Vliianie nizkochastotnoi elektricheskoi stimuliatsii zubchatogo iadra mozzhenka na ochagi epilepticheskoi aktivnosti [Effect of low-frequency electric stimulation of the dentate nucleus of the cerebellum on foci of epileptic activity]” Patologicheskaia Fiziologiia I Eksperimental'naia Terapiia 1989 May-June; (3):24-8). The authors further demonstrated that relatively low frequency (7-12 Hz) electrical stimulation of the dentate nucleus of the cerebellum induced seizure-related discharges in foci of epileptic activity produced in the brain cortex by application of penicillin solution. The authors additionally demonstrated that destruction of the ventrolateral nucleus of the thalamus abolished the effect of seizure discharge facilitation induced by stimulation of the dentate nucleus of the cerebellum. (Note that high frequency electrical stimulation of areas of the thalamus has been demonstrated to have inhibitory effects similar to a lesion.) In 1980, Heath, et al. demonstrated in monkeys that electrical stimulation through the vermis of the cerebellum inhibits epileptiform electroencephalographic activity at the cerebellum, septal region, and hippocampus (Heath, et al. “Feedback loop between cerebellum and septal-hippocampal sites: its role in emotion and epilepsy” Biological Psychiatry 1980 August; 15(4):541-56).

In 1992, Davis, et al. followed up 32 seizure patients who had undergone chronic cerebellar stimulation (CCS) since 1974 (Davis et al. “Cerebellar stimulation for seizure control: 17-year study.” Stereotactic and Functional Neurosurgery 1992; 58(1-4):200-8). The authors contacted 27 of these patients and found that nine (7 spastic, 2 epileptic) continued to use CCS for an average of 14.3 years (10-17 years). Six (67 percent) were seizure-free and three (33 percent) had a reduction of seizure frequency. Of two additional patients with spastic seizures who had used CCS for 13 years before their deaths, one had been seizure-free and the other had experienced a reduction. The remaining 16 patients (12 spastic, 4 epileptic) with nonfunctioning stimulators had used CCS for an average of 8.3 years (2-14 years); five (31 percent) continued to be seizure-free, seven (44 percent) had a reduction and four (25 percent) had no change or a slight increase. Overall, 23 (85 percent) patients benefitted from CCS. (Stimulation charge densities were 0.9-2.5° C./cm²/phase delivered at 10-180 pulses/sec to bilateral electrode pads on the superomedial cerebellar cortex.)

Direct electrical stimulation of the seizure focus may also be effective in the treatment of epilepsy. Velasco, et al. applied such therapy in patients with temporal lobe epilepsy (Velasco et al. “Subacute and chronic electrical stimulation of the hippocampus on intractable temporal lobe seizures. Preliminary report.” Archives of Medical Research 2000 May; 31(3):316-28). In each patient, depth electrodes were implanted in the hippocampus for purposes of verifying that the seizure focus was in or near the hippocampus. While the electrodes were implanted, electrical stimulation was applied for several weeks or months to the electrode(s) near the seizure focus. Most patients experienced a significant decrease in the number of daily seizures while such electrical stimulation was applied. Subsequent to verification of the location of the seizure focus, a portion of the temporal lobe containing the seizure focus was removed from most of these patients. Histopathology of the stimulated areas (i.e., areas near the electrodes) demonstrated no significant detrimental effects, and neuropsychological testing suggested only positive changes in memory due to stimulation.

As noted above, high frequency electrical stimulation has been as efficacious as a lesion in the same area, presumably by inhibiting neural activity in the area. Such inhibition may underlie the seizure reduction observed in direct stimulation of the seizure focus. Alternatively, neurostimulation at relatively lower frequencies may somehow activate and/or “re-program” local neural tissue, leading to reduced seizure activity. In contrast to ablation surgery, chronic electrical stimulation is reversible. Additionally, stimulation parameters may be adjusted to minimize side effects while maintaining efficacy; such “fine tuning” is unavailable when producing a lesion.

An implantable chronic stimulation device for DBS is commercially available and similar systems are under development. However, the current implant procedure is highly invasive, and the surgery for placement of the available system may require an entire day. These systems require the power source and stimulation electronics to be implanted far from the electrodes, generally in the chest or elsewhere in the trunk of the body. These bulky systems therefore require extensive invasive surgery for implantation, and breakage of the long leads is highly likely.

For instance, the system manufactured by Medtronic, Inc. of Minneapolis, Minn. has several problems that make it an unacceptable option for some patients. It requires a significant surgical procedure for implantation, as the implantable pulse generator (IPG), a major component of the system containing the stimulation electronics and power source, is implanted in the thorax and connected via a subcutaneous tunnel to an electrode through the chest, neck and head into the brain. The IPG is also bulky, which may produce an unsightly bulge at the implant site (e.g., the chest), especially for thin patients. Additionally, the system is powered by a primary battery, which lasts only 3-4 years under normal operation. When the battery ceases to provide sufficient energy to adequately power the system, the patient must undergo an additional surgery in order to replace the IPG.

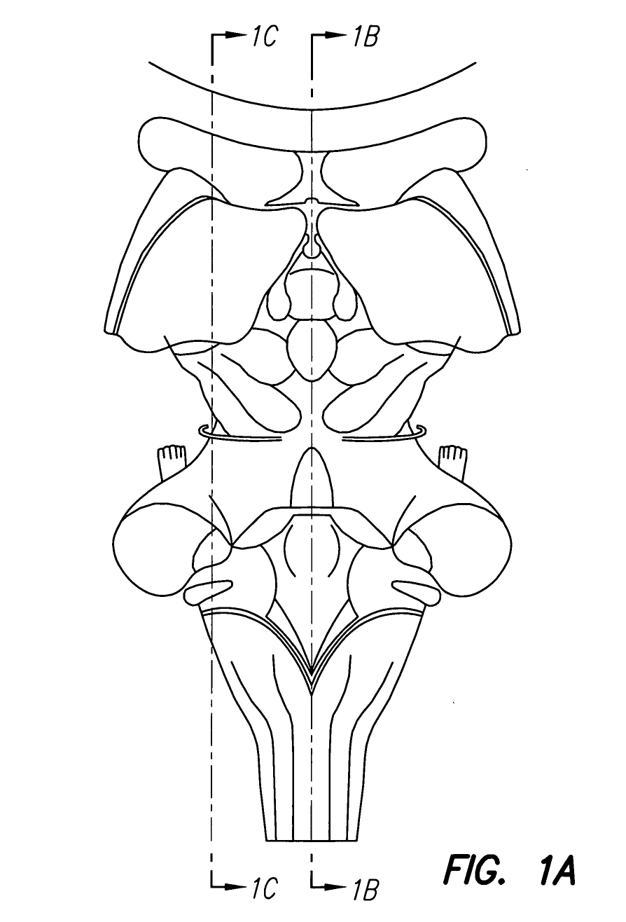

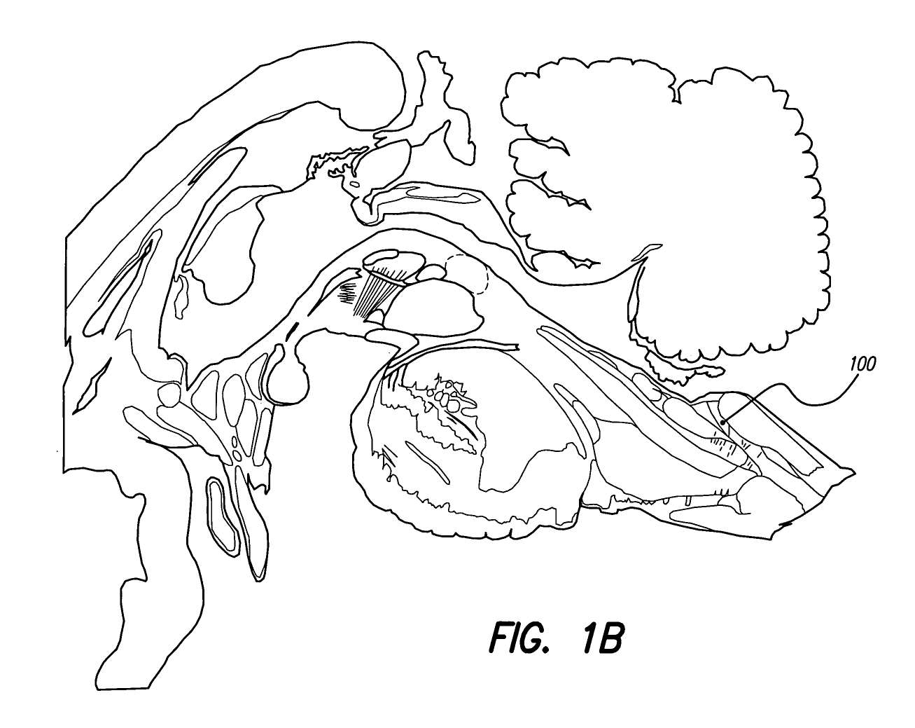

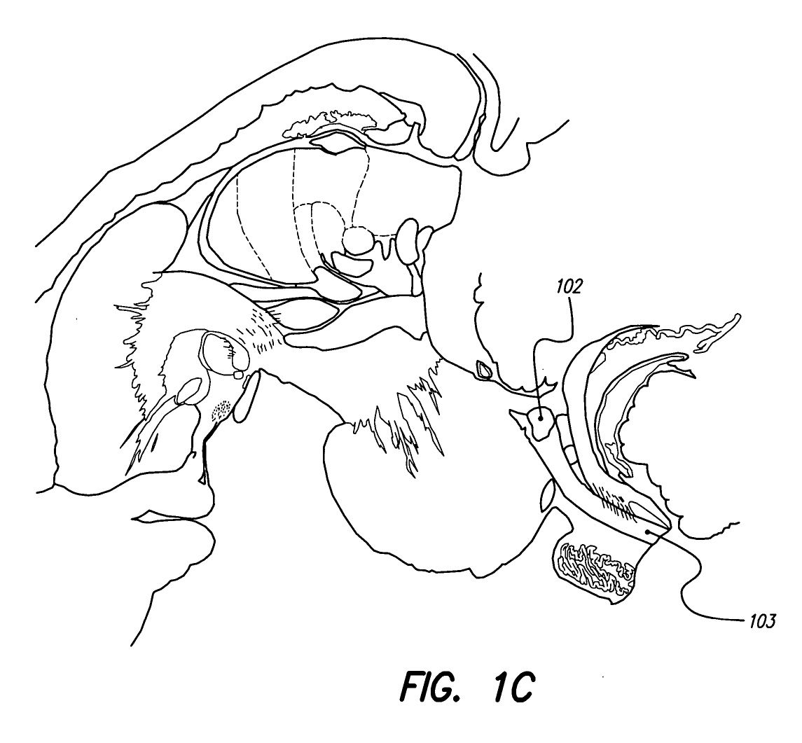

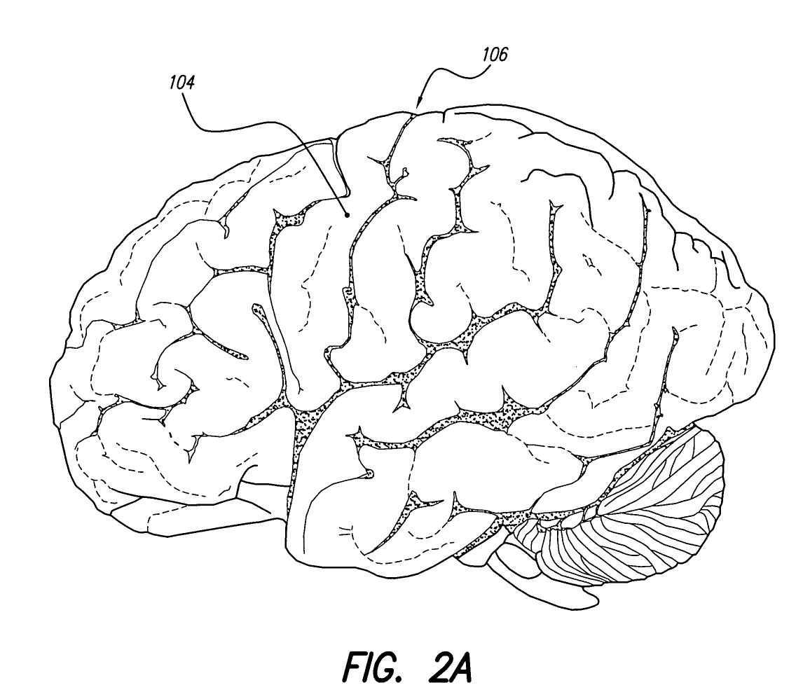

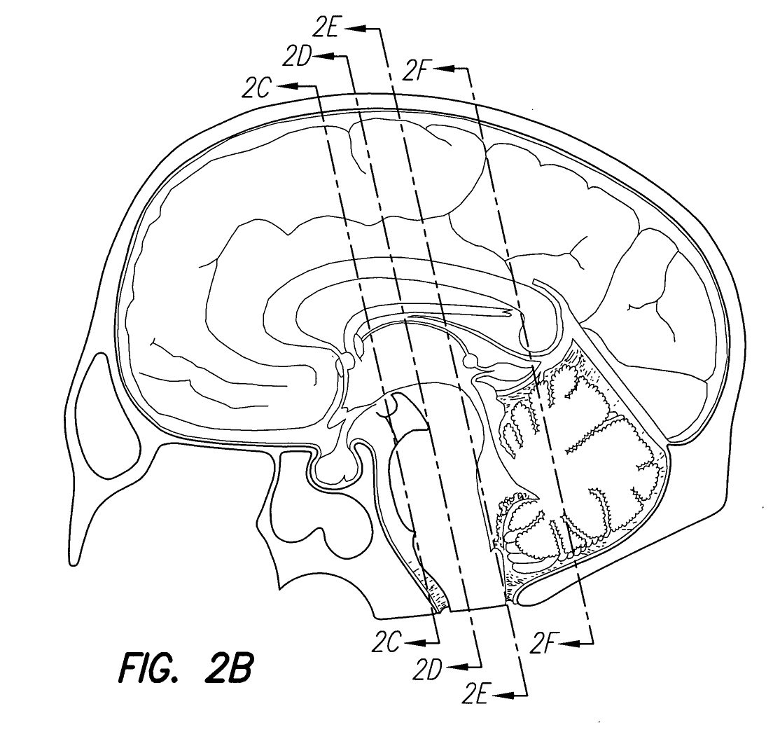

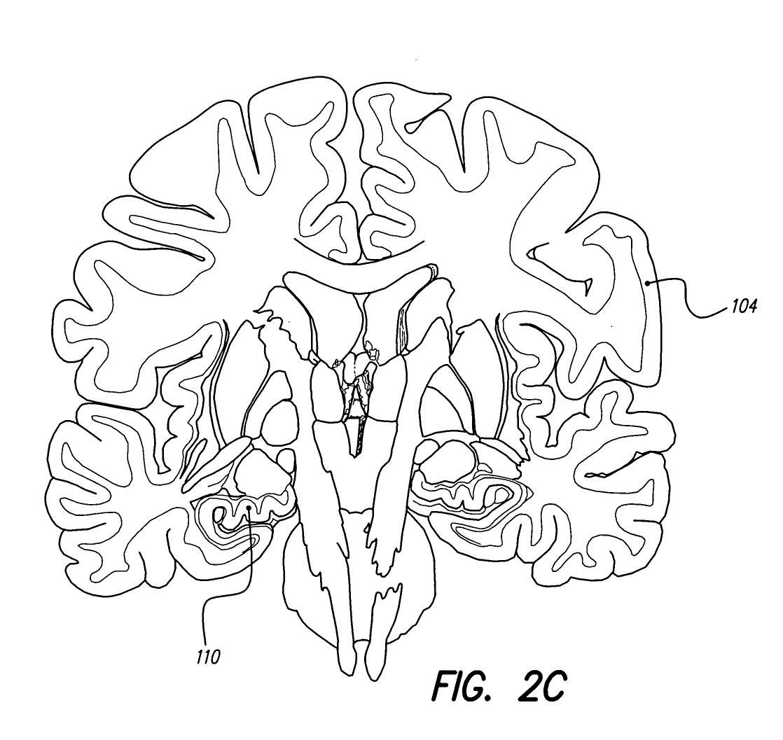

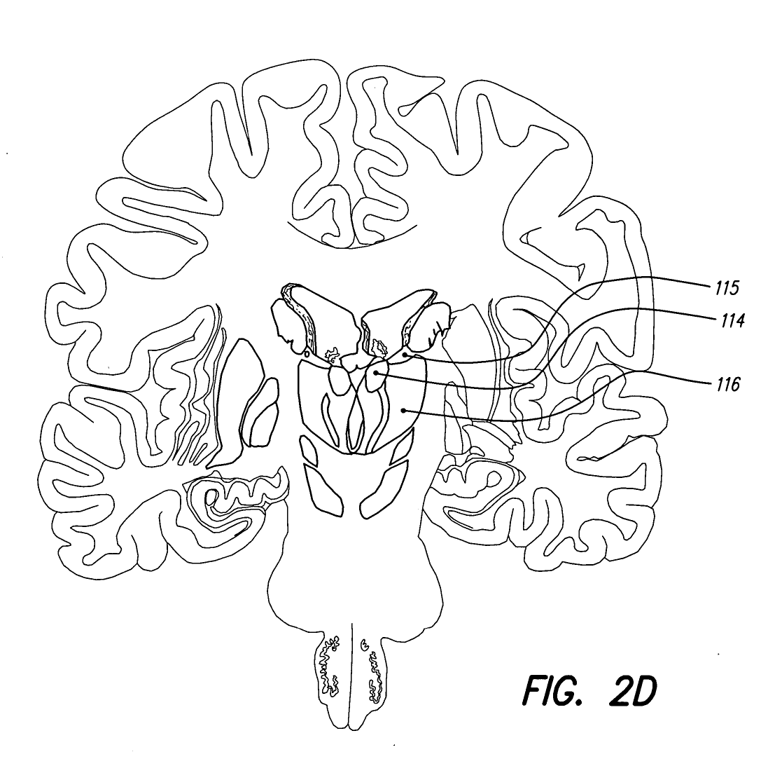

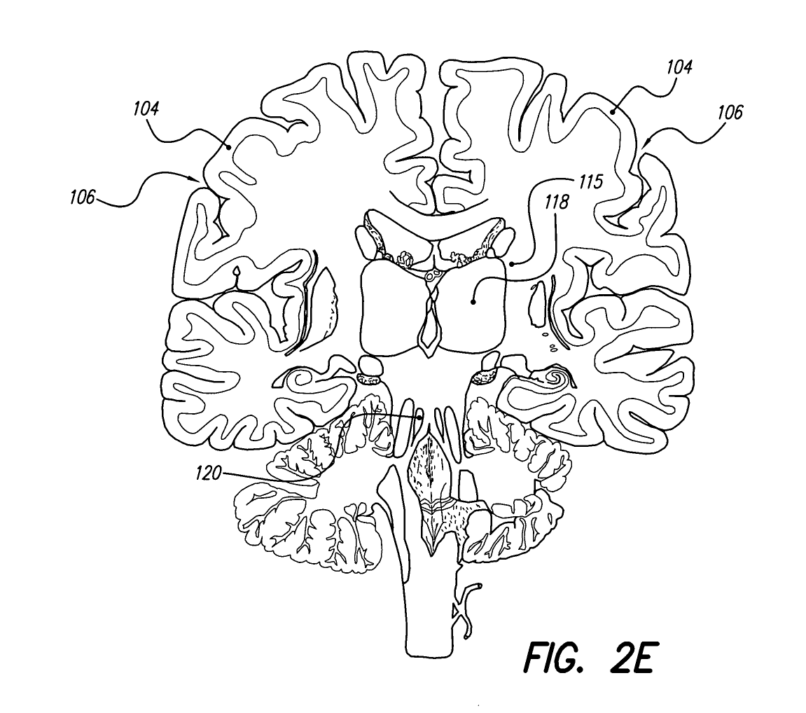

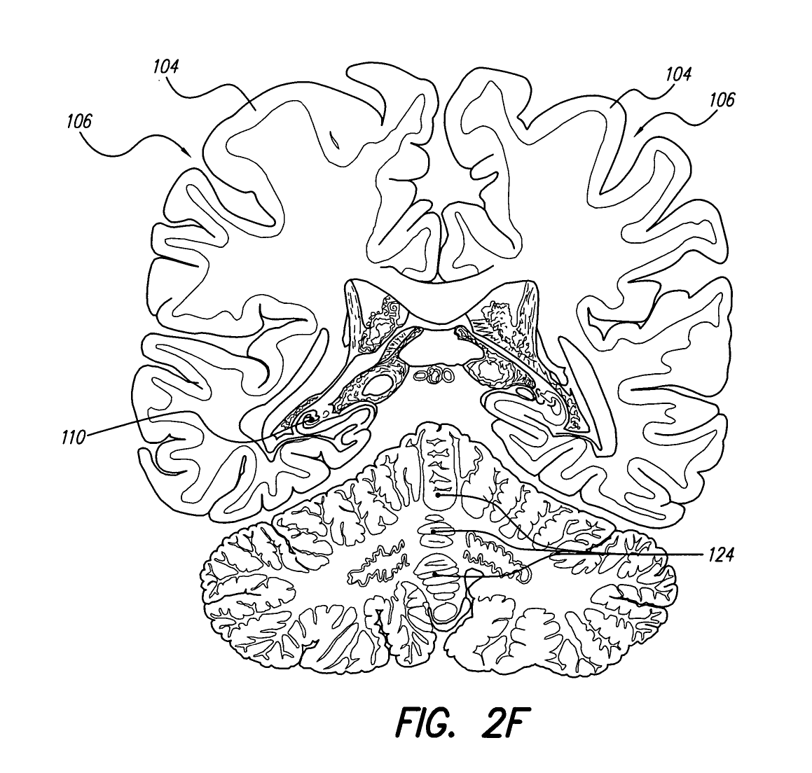

FIG. 1A depicts the dorsal surface of the brain stem, and FIGS. 1B and 1C are elongated cross-sectional views through the brain stem depicted in FIG. 1A. FIG. 2A depicts the lateral surface of the brain, FIG. 2B depicts the medial surface of the head, and FIGS. 2C-2F are coronal section views of the brain of FIG. 2B. FIG. 1B shows the location of the nucleus of the solitary tract (NTS) (100). FIG. 1C shows the principal (main) trigeminal sensory nucleus (102) and the spinal trigeminal nucleus (103). FIG. 2A shows the motor cortex (104) (which includes the precentral gyrus). As can be seen, the motor cortex (104) lies on the outermost region of the brain, along the top and sides of the skull, and is the most posterior portion of the frontal lobe, lying just anterior to the central sulcus (106) (also known as the central fissure). The motor cortex (104) is also shown in FIG. 2C, as is the hippocampus (110). FIG. 2D shows the thalamus (115) which includes the anterior nucleus (114) and the ventral lateral nucleus (116). The centromedian nucleus (118) of the thalamus (115) and the locus coeruleus (120) are shown in FIG. 2E. FIG. 2F shows the cerebellum, and again shows the motor cortex (104), central sulcus (106), and hippocampus (110).

In some embodiments, at least one stimulus is applied to a stimulation site within the brain of a patient to treat and/or prevent epilepsy. As used herein and in the appended claims, the term “stimulation site” refers to any nerve, organ, or other tissue within a patient to which at least one stimulus is applied to treat and/or prevent epilepsy. For example, the stimulation site may include, but is not limited to, a seizure focus, seizure foci, the thalamus (including centromedian, anterior, and ventrolateral nuclei and any other site of thalamic relay neurons), hippocampus, cerebellum, NTS, locus coeruleus, and mesial temporal lobe. The stimulation site may also include any nerve branching from the above-listed nerves. The stimulation site may also include any area of the brain that may propagate a seizure and/or any area in the brain that demonstrates increased activity in epileptics relative to non-epileptic controls.

The stimulus applied to the stimulation site may include electrical stimulation, also known as neuromodulation. The application of a high frequency electrical stimulation (i.e., greater than 400 Hz) to a stimulation site may force the neurons in the stimulation site to fire in an asynchronous manner and thereby prevent or abort a seizure. As mentioned, the primary pathology of epilepsy is a synchronization of electrical activity among large numbers of brain neurons. Neurons “fire”, i.e., transmit an electrical depolarization pulse down an axon(s), multiple times per second. While a group of adjacent neurons may normally demonstrate some correlation in their firing pattern, they normally do not all fire with exactly the same rate and exactly the same timing. However, during a seizure, a group of neurons in the brain demonstrates a highly synchronized firing pattern.

High frequency stimulation of the neurons in a stimulation site exploits the subtle physiological and anatomical differences between the neurons to cause the neurons to fire asynchronously. Each neuron in a stimulation site or population has slightly differing characteristics such as, but not limited to, absolute refractory periods, relative refractory periods, and resting membrane potentials. At relatively low stimulation frequencies, these differences are essentially unnoticeable, as most of the neurons recover before each stimulation pulse is applied. Thus, the neurons at low stimulation frequencies still appear to fire synchronously during a seizure. However, with stimulation at high frequencies, the subtle differences in neuron characteristics are accentuated. The firing rate of each neuron no longer depends on the specific rate of stimulation, but depends instead primarily on the characteristics of each individual neuron. Since these characteristics are slightly different for each neuron, the firing rates of the neurons follow a stochastic or asynchronous pattern. Thus, high frequency stimulation of a neuron population exploits subtle differences in the characteristics of each neuron to provide a pattern of firing that is stochastic and asynchronous, thereby preventing or aborting a seizure.

As used herein and in the appended claims, unless otherwise specifically denoted, “high frequency stimulation” refers to electrical stimulation having a frequency substantially equal to or greater than 400 Hz. The stimulation frequency may vary as best serves a particular application. For example, in some embodiments, the stimulation frequency be anywhere from 400 Hz to 5000 Hz or more.

In some embodiments, the high frequency stimulation is delivered to a stimulation site continuously. The high frequency stimulation may alternatively be delivered to a stimulation site periodically, semi-randomly, or randomly. The stimulation may be activated by the patient or a caretaker or it may be activated by a device that is implanted in the patient. For example, a sensing device may be configured to sense the occurrence or impending occurrence of a seizure and activate the high frequency stimulation accordingly.

The stimulus may additionally or alternatively include drug stimulation. Therapeutic dosages of one or more drugs may be infused into the stimulation site to treat and/or prevent epilepsy. The drugs may include, but are not limited to, gamma-aminobutyric acid (GABA) or a GABA agonist such as muscimol.

In some embodiments, the electrical stimulation and/or the drug stimulation may be performed by one or more implantable system control units (SCUs). As will be described in more detail below, an SCU may include an implantable signal generator coupled to one or more electrodes for delivering electrical stimulation to a stimulation site. The SCU may additionally or alternatively include an implantable pump coupled to one or more catheters for delivering drugs to a stimulation site.

The one or more SCUs may be small, implantable stimulators, referred to herein as microstimulators. The microstimulators of the present invention may be similar to or of the type referred to as BION® devices (Advanced Bionics® Corporation, Valencia, Calif.). The following documents describe various details associated with the manufacture, operation, and use of BION implantable microstimulators, and are all incorporated herein by reference in their respective entireties:

| Application/Patent/ | Filing/Publication | |
|---|---|---|
| Publication No. | Date | Title |
| U.S. Pat. No. 5,193,539 | Issued Mar. 16, 1993 | Implantable Microstimulator |
| U.S. Pat. No. 5,193,540 | Issued Mar. 16, 1993 | Structure and Method of Manufacture |
| | | of an Implantable Microstimulator |
| U.S. Pat. No. 5,312,439 | Issued May 17, 1994 | Implantable Device Having an |
| | | Electrolytic Storage Electrode |
| PCT Publication | Published Sep. 3, 1998 | Battery-Powered Patient Implantable |
| WO 98/37926 | | Device |
| PCT Publication | Published Oct. 8, 1998 | System of Implantable Devices For |
| WO 98/43700 | | Monitoring and/or Affecting Body |
| | | Parameters |
| PCT Publication | Published Oct. 8, 1998 | System of Implantable Devices For |
| WO 98/43701 | | Monitoring and/or Affecting Body |
| | | Parameters |
| U.S. Pat. No. 6,051,017 | Issued Apr. 18, 2000 | Improved Implantable |
| | | Microstimulator and Systems |
| | | Employing Same |
| | Published September, 1997 | Micromodular Implants to Provide |
| | | Electrical Stimulation of Paralyzed |
| | | Muscles and Limbs, by Cameron, et |
| | | al., published in IEEE Transactions on |
| | | Biomedical Engineering, Vol. 44, No. |
| | | 9, pages 781-790. |

The pump or controlled drug release device described herein may include any of a variety of different drug delivery systems. Controlled drug release devices based upon a mechanical or electromechanical infusion pump may be used. In other examples, the controlled drug release device can include a diffusion-based delivery system, e.g., erosion-based delivery systems (e.g., polymer-impregnated with drug placed within a drug-impermeable reservoir in communication with the drug delivery conduit of a catheter), electrodiffusion systems, and the like. Another example is a convective drug delivery system, e.g., systems based upon electroosmosis, vapor pressure pumps, electrolytic pumps, effervescent pumps, piezoelectric pumps and osmotic pumps.

Exemplary controlled drug release devices suitable for use as described herein include, but are not necessarily limited to, those disclosed in U.S. Pat. Nos. 3,760,984; 3,845,770; 3,916,899; 3,923,426; 3,987,790; 3,995,631; 4,016,880; 4,036,228; 4,111,202; 4,111,203; 4,203,440; 4,203,442; 4,210,139; 4,327,725; 4,360,019; 4,487,603; 4,627,850; 4,692,147; 4,725,852; 4,865,845; 4,911,616; 5,057,318; 5,059,423; 5,085,562; 5,112,614; 5,137,727; 5,219,278; 5,224,843; 5,234,692; 5,234,693; 5,271,724; 5,277,556; 5,728,396; 5,759,014; 5,759,015; 6,368,315; 6,464,687; 2004/0082908 and the like. All of these listed patents are incorporated herein by reference in their respective entireties.

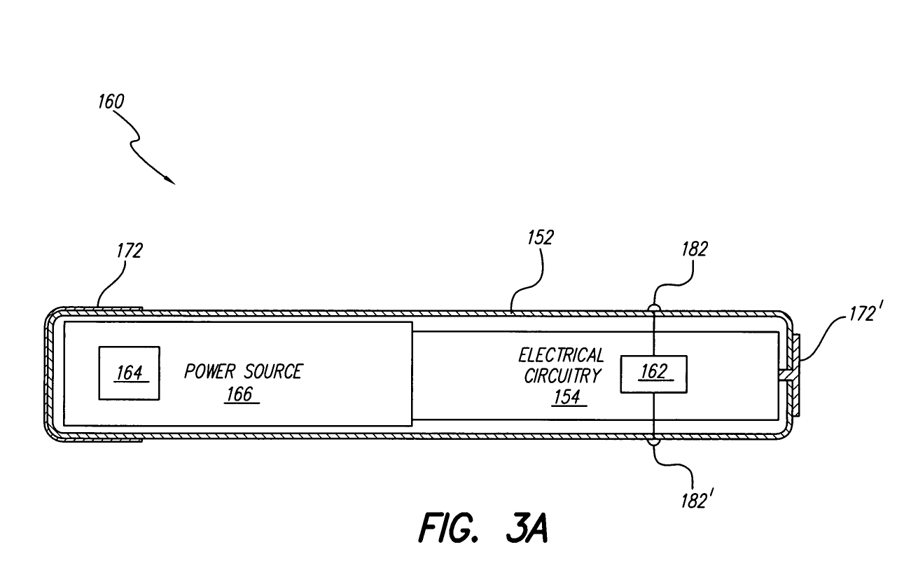

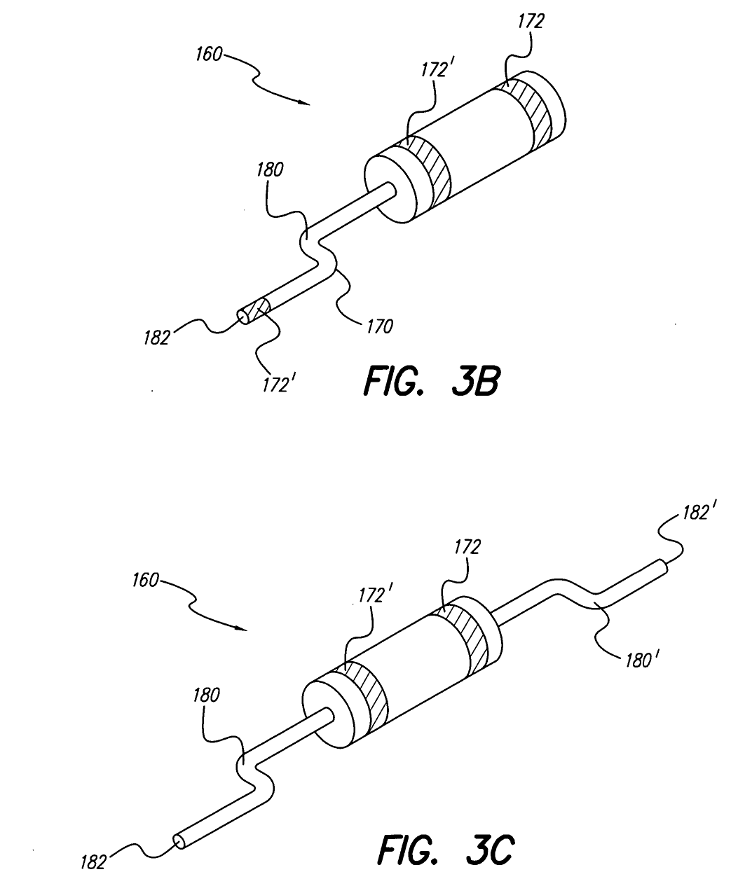

FIGS. 3A-3C illustrate an exemplary BION microstimulator SCU (160). As illustrated in FIG. 3A, the SCU (160) may include a number of components. The components may include, but are not limited to, a power source (166), electrical circuitry (154), a pump (162), and a programmable memory unit (164), all of which will be described in more detail below.

As shown in FIGS. 3A-3C, the microstimulator SCU (160) may include a narrow, elongated capsule or housing (152) containing electrical circuitry (154) connected to electrodes (172, 172′), which may pass through the walls of the capsule (152) at either end. The electrical circuitry (154) will be described in more detail below. Alternatively, the electrodes (172, 172′) may be built into the capsule (152) and/or arranged on a catheter (180; FIG. 3B) or at the end of a lead, as described below. As detailed in the referenced publications, electrodes (172, 172′) generally comprise a stimulating electrode (to be placed close to the target stimulation site) and an indifferent electrode (for completing the circuit). Other configurations of the microstimulator SCU (160) are possible, as is evident from the above-referenced publications, and as described in more detail herein.

The physical dimensions of the implantable microstimulator SCU (160) are sufficiently small to permit placement in or adjacent to the stimulation site that is to be stimulated. For example, the stimulator (160) may be a thin, elongated cylinder with a diameter of less than 5 mm and a length of less than about 25-35 mm. It will be recognized that the shape of the microstimulator (160) may be any shape as best serves a particular application and may be determined by the structure of the desired stimulation site, the surrounding area, and the method of implantation.

The microstimulator SCU (160) may be implanted within a patient with a surgical tool such as a hypodermic or bore needle or any other tool specially designed for the purpose. Alternatively, the microstimulator (160) may be implanted using endoscopic or laparoscopic techniques.

The external surfaces of the microstimulator SCU (160) may advantageously be composed of biocompatible materials. For example, the capsule (152) may be made of glass, ceramic, metal, or any other material that provides a hermetic package that will exclude water vapor but permit passage of electromagnetic fields used to transmit data and/or power. The electrodes (172, 172′; FIGS. 3B and 3C) may be made of a noble or refractory metal or compound, such as platinum, iridium, tantalum, titanium, titanium nitride, niobium or alloys of any of these, in order to avoid corrosion or electrolysis which could damage the surrounding tissues and the device.

In some embodiments, the microstimulator SCU (160) may include two leadless electrodes (172, 172′; FIGS. 3B and 3C). Either or both of the electrodes (172, 172′) may alternatively be located at the ends of short, flexible leads as described in U.S. patent application Ser. No. 09/624,130, filed Jul. 24, 2000, which is incorporated herein by reference in its entirety. The use of such leads permits, among other things, electrical stimulation to be directed more locally to targeted tissue(s) a short distance from the surgical fixation of the bulk of microstimulator SCU (160), while allowing most elements of the microstimulator (160) to be located in a more surgically convenient site. This minimizes the distance traversed and the surgical planes crossed by the microstimulator (160) and any lead(s).

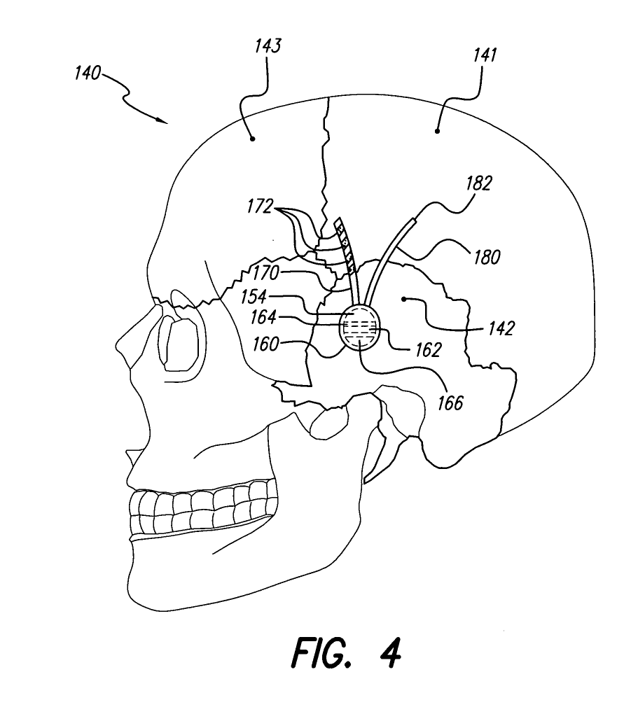

FIG. 4 shows an SCU (160) that has been implanted beneath the scalp of a patient. The SCU 160 may be implanted in a surgically-created shallow depression or opening in the skull (140). For instance, the depression may be made in the parietal bone (141), temporal bone (142), frontal bone (143), or any other bone within the skull (140) as best serves a particular application. The SCU (160) may conform to the profile of surrounding tissue(s) and/or bone(s), thereby minimizing the pressure applied to the skin or scalp.

As shown FIG. 4, the SCU (160) may be coupled to one or more electrode leads (170) and/or catheters (180). The lead (170) may include the electrodes (172) and may contain insulated wires electrically coupling the electrodes (172) to the SCU (160). The one or more electrode leads (170) and/or catheters (180) may run subcutaneously, for instance, in a surgically-created shallow groove(s) in the skull, to an opening(s) in the skull, and pass through the opening(s) into or onto the desired stimulation site within the brain. Such recessed placement of the SCU (160) and the lead(s) (170) and/or catheter(s) (180) may decrease the likelihood of erosion of the overlying skin, and may minimize any cosmetic impact.

As shown in FIG. 4, the SCU (160) includes electrical circuitry (154) configured to produce electrical stimulation pulses that are delivered to the stimulation site via the electrodes (172). In some embodiments, the electrical circuitry (154) is configured to produce monopolar stimulation. The electrical circuitry (154) may alternatively or additionally be configured to produce bipolar stimulation. The electrical circuitry (154) may include one or more processors configured to decode stimulation parameters and generate the stimulation pulses. The electrical circuitry (154) may also include an inductive coil for receiving and transmitting RF data and/or power. The electrical circuitry (154) may include additional circuitry such as capacitors, integrated circuits, resistors, coils, and the like configured to perform a variety of functions as best serves a particular application.

The electrical stimulation may alternatively be provided as described in International Patent Application Serial Number PCT/US01/04417 (the '417 application), filed Feb. 12, 2001, and published Aug. 23, 2001 as WO 01/60450, which application is incorporated herein by reference in its entirety. As such, the electrical stimulation of the present invention may be as provided according to the manner described in this PCT application, which is directed to a “Deep Brain Stimulation System for the Treatment of Parkinson's Disease or Other Disorders”.

As shown in FIG. 4, a pump (162) may also be included within the SCU (160). The pump (162) is configured to store and dispense one or more drugs through one or more infusion outlets (182, 182′; FIG. 3A) via the catheter (180).

The SCU (160) may also include a power source (166). The power source (166) may be a primary battery, a rechargeable battery, a capacitor, or any other suitable power source.

The SCU (160) may also include a programmable memory unit (164) for storing one or more sets of data and/or stimulation parameters. The stimulation parameters may include, but are not limited to, electrical stimulation parameters and drug stimulation parameters. The programmable memory (164) allows a patient, clinician, or other user of the SCU (160) to adjust the stimulation parameters such that the electrical stimulation and/or drug stimulation are at levels that are safe and efficacious for a particular nerve injury and/or for a particular patient. Specific stimulation parameters may provide therapeutic advantages for various types of epilepsy. Thus, stimulation parameters may be chosen to target specific neural populations and/or to exclude others, or to increase neural activity in specific neural populations and/or to decrease neural activity in others. The electrical stimulation and drug stimulation parameters may be controlled independently. However, in some instances, the electrical stimulation and drug stimulation parameters are coupled, e.g., electrical stimulation may be programmed to occur only during drug stimulation. The programmable memory (164) may be any type of memory unit such as, but not limited to, random access memory (RAM), static RAM (SRAM), a hard drive, or the like.

The electrical stimulation parameters may control various parameters of the stimulation current applied to a nerve including, but not limited to, the frequency, pulse width, and amplitude of the stimulation current. The drug stimulation parameters may control various parameters including, but not limited to, the amount of drugs infused into the stimulation site, the rate of drug infusion, and the frequency of drug infusion.

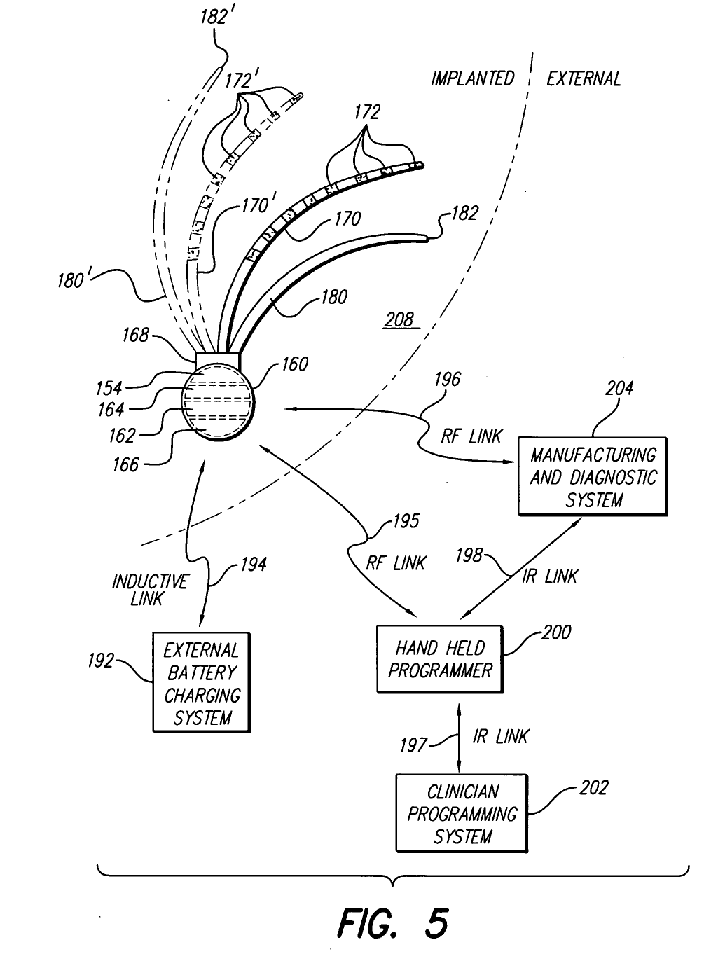

FIG. 5 shows that the implanted SCU (160) may be configured to communicate with a number of external devices. For example, an external battery charging system (EBCS) (192) may provide power used to recharge the power source (166) via an RF link (194). External devices including, but not limited to, a hand held programmer (HHP) (200), clinician programming system (CPS) (202), and/or a manufacturing and diagnostic system (MDS) (204) may be configured to activate, deactivate, program, and test the SCU (160) via one or more RF links (196, 195). One or more of these external devices (204, 200, 202) may also be used to control the delivery of one or more drugs to the stimulation site. The CPS (202) may communicate with the HHP (200) via an infrared (IR) link (197) or via any other suitable communication link. Likewise, the MDS (204) may communicate with the HHP (200) via an IR link (198) or via any other suitable communication link.

The HHP (200), MDS (204), CPS (202), and EBCS (192) are merely illustrative of the many different external devices that may be used in connection with the SCU (140). Furthermore, it will be recognized that the functions performed by the HHP (200), MDS (204), CPS (202), and EBCS (192) may be performed by a single external device. One or more of these external devices (192, 200, 202, 204) may be embedded in a seat cushion, mattress cover, pillow, garment, belt, strap, pouch, or the like.

The SCU (160) may be configured to operate independently. Alternatively, the SCU (160) may be configured to operate in a coordinated manner with one or more additional SCUs (160), other implanted devices, or other devices external to the patient's body. For instance, a first SCU (160) may control or operate under the control of a second SCU (160), other implanted device, or other device external to the patient's body. The SCU (160) may be configured to communicate with other implanted SCUs (160), other implanted devices, or other devices external to the patient's body via an RF link, an untrasonic link, an optical link, or any other type of communication link. For example, the SCU (160) may be configured to communicate with an external remote control that is capable of sending commands and/or data to the SCU (160) and that is configured to receive commands and/or data from the SCU (160).

The SCU (160) may include one or more sensing devices configured to sense a patient's response to and/or need for treatment. For example, the sending devices may be configured to sense the occurrence of a seizure, impending occurrence of a seizure, or symptoms thereof. These symptoms may include, but are not limited to, electrical activity of the brain (e.g., EEG or discharge frequency of a neural population), patient response to medication, neurotransmitter levels, hormone levels, and/or any other activity related to epilepsy. The SCU (160) may alternatively or additionally be configured to communicate with one or more separate sensing devices configured to sense a patient's response to and/or need for treatment. The sensed information may be used to help determine the strength and/or duration of electrical stimulation and/or the amount and/or type(s) of stimulating drug(s) required to produce the desired effect. Alternatively, the SCU (160) may not include any sensing devices.

Thus, it is seen that one or more external appliances may be provided to interact with the SCU (160), and may be used to accomplish at least one or more of the following functions:

Function 1: If necessary, transmit electrical power to the SCU (160) in order to power the SCU (160) and/or recharge the power source (166).

Function 2: Transmit data to the SCU (160) in order to change the stimulation parameters used by the SCU (160).

Function 3: Receive data indicating the state of the SCU (160) (e.g., battery level, drug level, stimulation parameters, etc.).

Additional functions may include adjusting the stimulation parameters based on information sensed by the SCU (160) or by other sensing devices.

By way of example, an exemplary method of treating epilepsy may be carried out according to the following sequence of procedures. The steps listed below may be modified and/or added to as best serves a particular application.

1. An SCU (160) is implanted so that its electrodes (172) and/or infusion outlet (182) are located in or on or near a stimulation site.

2. The SCU (160) is programmed to apply at least one stimulus to the stimulation site. The stimulus may include a high frequency electrical stimulation and/or drug stimulation.

3. When the patient desires to invoke electrical and/or drug stimulation, the patient sends a command to the SCU (160) (e.g., via a remote control) such that the SCU (160) delivers the prescribed electrical and/or drug stimulation. The SCU (160) may be alternatively or additionally configured to automatically apply the electrical and/or drug stimulation in response to the occurrence or impending occurrence of a seizure.

4. To cease electrical and/or drug stimulation, the patient may turn off the SCU (160) (e.g., via a remote control).

5. Periodically, the power source (166) of the SCU (160) is recharged, if necessary, in accordance with Function 1 described above.

For the treatment of any of the various types of epilepsy, it may be desirable to modify or adjust the algorithmic functions performed by the implanted and/or external components, as well as the surgical approaches. For example, in some situations, it may be desirable to employ more than one SCU (160), each of which could be separately controlled by means of a digital address. Multiple channels and/or multiple patterns of electrical and/or drug stimulation might thereby be used to deal with complex or multiple symptoms or conditions, such as temporal lobe epilepsy attributed to bilateral mesial temporal sclerosis.

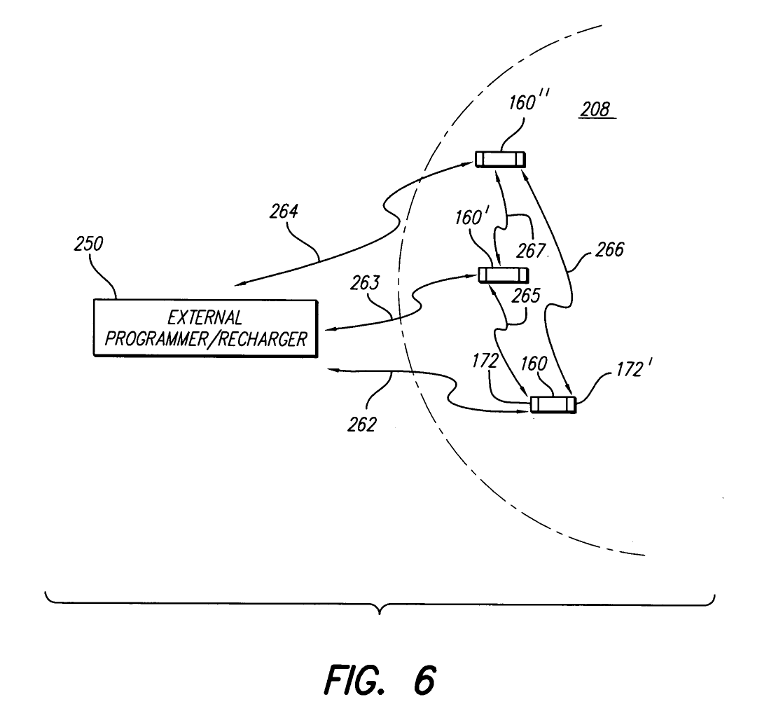

For instance, as shown in the example of FIG. 6, a first SCU (160) implanted beneath the skin of the patient (208) provides a first medication or substance; a second SCU (160′) provides a second medication or substance; and a third SCU (160″) provides electrical stimulation via electrodes (172, 172′). As mentioned earlier, the implanted devices may operate independently or may operate in a coordinated manner with other similar implanted devices, other implanted devices, or other devices external to the patient's body, as shown by the control lines (262-267) in FIG. 6. That is, an external controller (250) may be configured to control the operation of each of the implanted devices (160, 160′, and 160″). In some embodiments, an implanted device, e.g. SCU (160), may control or operate under the control of another implanted device(s), e.g. SCU (160′) and/or SCU (160″).

The preceding description has been presented only to illustrate and describe embodiments of the invention. It is not intended to be exhaustive or to limit the invention to any precise form disclosed. Many modifications and variations are possible in light of the above teaching.

1. A method of treating a patient with epilepsy, said method comprising:
applying a stimulus to a stimulation site within said patient with an implanted system control unit in accordance with one or more stimulation parameters;
wherein said stimulus comprises a stimulation current delivered via one or more electrodes of said system control unit, said stimulation current having a frequency substantially equal to or greater than 400 Hertz (Hz).

2. The method of claim 1, wherein said frequency of said stimulation current is substantially equal to or greater than 400 Hz and substantially equal to or less than 5000 Hz.

3. The method of claim 1, wherein said stimulation current is configured to cause a group of neurons within said stimulation site to fire asynchronously.

4. The method of claim 1, wherein said system control unit is coupled to at least one catheter, and wherein said stimulus comprises stimulation via one or more drugs delivered through said at least one catheter.

5. The method of claim 1, wherein said stimulation parameters control one or more of a frequency of said stimulation current, a pulse width of said stimulation current, and an amplitude of said stimulation current.

6. The method of claim 1, further comprising sensing at least one condition related to epilepsy and using said at least one sensed condition to automatically adjust one or more of said stimulation parameters.

7. The method of claim 6, wherein said at least one sensed condition is at least one or more of an electrical activity of a brain of said patient, a hormone level, a neurotransmitter level, a response of said patient to a medication, and a response of said patient to said stimulus.

8. The method of claim 1, further comprising manually adjusting said stimulation parameters.

9. The method of claim 1, wherein said system control unit comprises a micro stimulator.

10. A system for treating a patient with epilepsy, said system comprising:
a system control unit configured to apply a stimulus to a stimulation site within said patient in accordance with one or more stimulation parameters, said system control unit comprising one or more electrodes;
wherein said system control unit and said electrodes are implanted within said patient and wherein said stimulus comprises a stimulation current delivered via said electrodes, said stimulation current having a frequency substantially equal to or greater than 400 Hertz (Hz).

11. The system of claim 10, wherein said frequency of said stimulation current is substantially equal to or greater than 400 Hz and substantially equal to or less than 5000 Hz.

12. The system of claim 10, wherein said stimulation current is configured to cause a group of neurons within said stimulation site to fire asynchronously.

13. The system of claim 10, further comprising a pump for delivering one or more drugs to said stimulation site, said pump coupled to a catheter, and wherein said stimulus comprises stimulation via said one or more drugs delivered through said catheter.

14. The system of claim 10, wherein said stimulation parameters control one or more of a frequency of said stimulation current, a pulse width of said stimulation current, and an amplitude of said stimulation current.

15. The system of claim 10, further comprising:
a sensor device for sensing at least one condition related to epilepsy;
wherein said system control unit uses said at least one sensed condition to automatically adjust one or more of said stimulation parameters.

16. The system of claim 15, wherein said at least one sensed condition is at least one or more of an electrical activity of a brain of said patient, a hormone level, a neurotransmitter level, a response of said patient to a medication, and a response of said patient to said stimulus.

17. The system of claim 10, wherein said system control unit comprises a microstimulator.

18. A system for treating a patient with epilepsy, said system comprising:
means for applying a stimulus to a stimulation site within said patient with an implanted system control unit in accordance with one or more stimulation parameters;
wherein said stimulus comprises a stimulation current delivered via one or more electrodes, said stimulation current having a frequency substantially equal to or greater than 400 Hertz (Hz).

19. The system of claim 18, wherein said system control unit is coupled to at least one catheter, and wherein said stimulus comprises stimulation via one or more drugs delivered through said at least one catheter.

20. The system of claim 18, further comprising means for sensing at least one condition related to epilepsy and using said at least one sensed condition to automatically adjust one or more of said stimulation parameters.
